(19) United States
(12) Patent Application Publication
(10) Pub. No.: US 2011/0301098 A1
(43) Pub. Date: Dec. 8, 2011

(54) TREATMENT FOR INTIMAL HYPERPLASIA AND RELATED CONDITIONS

(76) Inventors: Paul H. A. Quax, Leiden (NL); Abbey Schepers, Leiden (NL)

(21) Appl. No.: 12/374,291

(22) PCT Filed: Jul. 20, 2007
(86) PCT No.: PCT/AU2007/001008

§ 371 Date: Jun. 23, 2011

(30) Foreign Application Priority Data
Jul. 21, 2006 (AU) 2006903938

(51) Int. Cl. A61K 38/12; A61P 9/00

(52) U.S. Cl. 514/21.1

(57) ABSTRACT

The present invention provides a method of prevention or treatment of intimal hyperplasia in blood vessel walls, the method comprising the step of administering a therapeutically effective amount of an inhibitor of C5a function to a mammal.

### FIELD OF THE INVENTION

This invention relates to methods and compositions for the prevention of invasion of cells into materials implanted into the body. In particular it relates to the prevention of failure of blood vessel grafts as a result of restenosis caused by intimal hyperplasia and accelerated atherosclerosis, and to the prevention of occlusion of implants or prostheses caused by intimal hyperplasia.

### BACKGROUND OF THE INVENTION

Atherosclerosis is the most common form of vascular disease, and leads to insufficient blood supply to critical body organs, resulting in heart attack, stroke, and kidney failure. Atherosclerosis also causes major complications in tobacco smokers and in people suffering from hypertension, metabolic syndrome or diabetes. Atherosclerosis is a form of chronic vascular injury in which some of the normal vascular smooth muscle cells (“VSMC”) in the artery wall, which ordinarily control the vascular tone which regulates blood flow, change their nature and develop cancer-like behaviour. These VSMC become abnormally proliferative, and secrete substances such as growth factors, tissue degradation enzymes and other proteins, which enable them to invade and spread into the inner vessel lining, blocking blood flow and making that vessel abnormally susceptible to being completely blocked by local blood clotting, ultimately resulting in the death of the tissue served by that artery.

Vein bypass grafting is the most common method of vascular reconstruction to treat obstructive arterial lesions. Autologous vein grafts remain the only surgical alternative for many types of vascular reconstruction, but the failure rate of these grafts after 1 year approaches 20%, with the failure rate of aorta-coronary and peripheral vein grafts reaching 10% to 40% after 1 year and 50% to 60% after 10 years. The development of neointima or arteriosclerosis in the grafted vessel often leads to obliterative stenosis. The hallmarks of neointimal lesions are mononuclear cell infiltration, smooth muscle cell proliferation, and extracellular matrix deposition. The pathogenesis of this disease remains poorly understood, and no successful clinical interventions have been identified.

In patients with angiographic evidence of occlusive disease after vein grafting, atherosclerotic lesions within the graft have been demonstrated histologically as early as 6 to 12 months after surgery. The structural changes in venous bypass grafts result from the development of a rapidly progressive and structurally distinct form of atherosclerosis, which is generally known as accelerated atherosclerosis and is morphologically different from native atherosclerosis. Vein graft atherosclerotic lesions are more diffuse, concentric, and friable, with a poorly developed or absent fibrous cap, whereas native vessel atheromata are proximal, focal eccentric, and non-friable, with a well-developed fibrous cap. Accelerated atherosclerotic lesions also contain more foam cells, with varying degrees of lipid accumulation and macrophage/mononuclear and inflammatory cell infiltration, than native atherosclerotic lesions.

Restenosis, the recurrence of stenosis or artery stricture after corrective surgery, is an accelerated form of atherosclerosis. Recent evidence has supported a unifying hypothesis of vascular injury, in which coronary artery restenosis, coronary vein graft and cardiac allograft atherosclerosis can be considered to represent a much-accelerated form of the same pathogenic process which results in spontaneous atherosclerosis (Ip et al. (1990) J Am Coll Cardiol 15: 1667-1687; Muller et al. (1992) J Am Coll Cardiol 19: 418-432).

It has been suggested that restenosis is due to a complex series of fibroproliferative responses to vascular injury, involving potent growth-regulatory molecules, including platelet-derived growth factor (PDGF) and basic fibroblast growth factor (bFGF) which are also common to the later stages in atherosclerotic lesions, resulting in vascular smooth muscle cell proliferation, migration and neointimal accumulation.

Restenosis occurs after coronary artery bypass surgery, endarterectomy, and heart transplantation, and particularly after cardiac balloon angioplasty, atherectomy, laser ablation or endovascular stenting, in each of which one-third of patients redevelop restenosis by 6 months after surgery, and is responsible for recurrence of symptoms or death. Repeat revascularization surgery is often required. Despite over a decade of research and significant improvements in the primary success rate of the various medical and surgical treatments of atherosclerotic disease, including angioplasty, bypass grafting and endarterectomy, secondary failure due to late restenosis continues to occur in 30-50% of patients.

Vascular stenosis is also a major limitation of long-term success of organ transplants, eventually resulting in ischemic graft failure. Although the pathogenesis of this phenomenon, known as allograft-accelerated transplant sclerosis is not completely understood, it does involve endothelial damage, mononuclear cell infiltration, smooth muscle cell proliferation, and deposition of matrix protein in the intima. Several mouse models for this syndrome are available (Xu (2004) American Journal of Pathology 165(1): 1-10).

The development of early intimal thickening as a result of intimal hyperplasia (IH) and accelerated atherosclerosis is believed to be the result of an inflammatory reaction in the vein graft wall, initiated by mechanical damage during surgery, cyclic stretching and a higher shear stress in the arterial circulation. Both of these phenomena may contribute to eventual graft failure.

Intimal hyperplasia, also known as neointimal hyperplasia, is the abnormal migration and proliferation of vascular smooth muscle cells in the intimal layer of blood vessels, with associated deposition of extracellular connective tissue matrix.

One of the first phenomena to occur in this remodelling process is chemotaxis, followed by adhesion and migration of inflammatory eel k, predominantly monocytes, into the intima of the vein graft wall. These cells are the source of pro-inflammatory cytokines and growth factors, which are potent stimuli for smooth muscle cell migration and foam cell accumulation. The accumulation of smooth muscle cells and foam cells in the intima of the vein graft results in a thickened vessel wall and a reduced luminal diameter.

Drug-coated or drug-eluting stents which incorporate agents such as taxol, picrolimus, rapamycin and the like have been widely used to try to overcome the problem of reocclusion of stents by the ingrowth of cells from the intimal layer of the host artery (Bhargava et al. (2003) British Medical Journal 327: 274-279). Although these drug-eluting stents have met with some success, they primarily inhibit invasion of VSMCs. They do not address the overall problem of intimal hyperplasia.

Pexeluzimab (Alexion Pharmaceuticals, Inc.), a humanised monoclonal single-chain antibody fragment directed against C5, has been tested as an adjunctive therapy to thrombolytic agents or primary percutaneous transluminal coronary angioplasty (PCA) in patients suffering from acute myocardial infarctions. No benefit was observed for pexeluzimab in conjunction with thrombolytics, and although there was a reduction in mortality in the PCA group, there was no reduction in infarct size (Granger et al. (2003) Circulation 108: 1184-1190; Shernan et al. (2004)Ann. Thorac. Surg. 77: 942-949). In another study on patients undergoing coronary artery bypass grafts (Verrier et al. (2004) JAMA 291: 2319-2327), pexeluzimab did not reduce the risk of death or myocardial infarction inpatients undergoing bypass grafts only, but did reduce some risk in patients undergoing grafts with or without valve surgery. However, to our knowledge it has not been suggested that either pexeluzimab or eculizamab (another anti-C5 antibody produced by Alexion) might be useful in inhibition of intimal hyperplasia.

There is a need for a successful chemotherapeutic therapy to reduce or prevent vascular blockage. The most effective way to prevent this disease is at the cellular level, as opposed to repeated revascularization surgery, which can carry a significant risk of complications or death, consumes time and money, and is inconvenient to the patient.

### SUMMARY OF THE INVENTION

Inhibition of the chemotactic and cell activation processes involved in intimal hyperplasia could potentially result in reduced inflammatory cell adhesion, influx and activation, and ultimately in reduced vein graft thickening and stent restenosis. A variety of growth factors, such as platelet-derived growth factor (PDGF) and chemokines, and other targets such as certain G-protein coupled receptors, have been implicated in the actiology of intimal hyperplasia, but the mechanism of this condition is still not understood.

C5a, one of the biologically active components of the complement cascade, is a potent chemotactic protein. The complement cascade is an important part of the immune system, and consists of a group of circulating proteins and several membrane-bound regulatory enzymes. Activation of the complement cascade leads to the cleavage of complement component C3, which in turn results in the formation of biologically active end-products, including C5a.

C5a exerts its function via the C5a receptor (C5aR) and is chemotactic for numerous cell types, including monocytes, T- and B-lymphocytes, and neutrophils. C5a has been shown to be important in several inflammatory processes, such as sepsis, bronchial asthma and ischemia/reperfusion injury. However, the role, if any, of C5a in vascular inflammatory processes such as atherosclerosis and post-interventional vessel remodelling in situations such as post-angioplasty restenosis and vein graft thickening is still not fully understood, and hitherto there have not been any specific studies on this point.

We hypothesized that C5a plays a pro-inflammatory, prostenotic role in vein graft disease, and that interference in C5a function should lead to a reduction in intimal hyperplasia and a decreased number of macrophage-derived foam cells in the intimal hyperplasia. We provide here, for the first time, evidence that C5a plays a functional role in the development of vein graft thickening, and that blockade of C5a is a potential target for therapy in order to overcome vein graft failure and stent restenosis.

In a first aspect, the invention provides a method of prevention or treatment of intimal hyperplasia in blood vessel walls, comprising the step of administering a therapeutically effective amount of an inhibitor of C5a function to a mammal.

In a second aspect, the invention provides a method of inhibiting the development of blood vessel graft thickening, comprising the step of administering a therapeutically effective amount of an inhibitor of C5a function to a mammal.

In a third aspect, the invention provides a method of inhibiting the development of stent restenosis, comprising the step of administering a therapeutically effective amount of an inhibitor of C5a function to a mammal into which a stent has been implanted.

In a fourth aspect, the invention provides a method of preventing or treating stenosis, restenosis or unwanted proliferation, migration or hypertrophy of cells in blood vessel walls or other anatomical structures of a mammal, comprising the step of administering a therapeutically effective amount of an inhibitor of C5a function to the mammal.

The stenosis, restenosis or unwanted proliferation, migration or hypertrophy of cells in blood vessel walls or other anatomical structures of a mammal may be a result of one of the following list of conditions; atherosclerosis, chronic obstructive pulmonary disorder, transplantation, vascular graft, venous surgery, arterial surgery, bypass graft failure, plastic surgery, tissue grafting, tumours, macular degeneration, neovascularisation, aberrant wound repair, endometriosis, vasculitis, defective revascularisation following thrombosis, prosthetic surgery, scarring, aneurysm surgery/repair, lymphatic surgery/repair, spinal injury/surgery/repair, endothelial tumours, cheloids, granulomas, haemangiomas, treatment/repair of post thrombotic disorders, angioplasty and reconstruction procedures.

### BRIEF DESCRIPTION OF THE FIGURES

FIG. 1 shows the structures of preferred cyclic peptide C5a receptor antagonists for use in the invention.

FIG. 2 illustrates the detection of C5 in venous bypass grafts by immunohistochemistry at different times after surgery. Directly after surgery, C5 is seen in adhering leucocytes. The highest amounts of C5 are seen 7 days after surgery, and at later time-points C5 remains present predominantly in endothelial cells, foam cells and adventitial fibroblasts (magnification 150-400×).

FIG. 3 shows the expression of C5a receptor mRNA in vein grafts as detected by semi-quantitative RT-PCR, compared with basal expression in caval veins (n=4 per time point). Very small amounts of C5a receptor mRNA are present in caval veins. An up-regulation of C5a receptor mRNA is seen after interposition of the graft, with peak expression 7 days after surgery, after which the level of expression returns to normal.

FIG. 4 shows the effect of application of C5a to vein grafts in hypercholesterolaemic mice (n=8 per group).

Panel A: Representative cross-sections of vein grafts exposed to dither 20% Pluronic gel alone, 20% Pluronic get containing 0.5 μg recombinant C5a protein or 20% Pluronic gel containing 5 μg recombinant C5a protein.

Panel B: A dose-dependent increase in intimal hyperplasia was seen in the C5a-treated mice (0.5 μg: p=0.1, 5 μg: p=0.002; arrows indicate intimal hyperplasia; magnification 200×).

FIG. 5 illustrates the effect of application of C5a to vein grafts on the foam cell contribution to intimal hyperplasia.

Panel A: Increased exposure to C5a results in a significant increase in foam cell contribution to intimal hyperplasia, as assessed with immunohistochemistry using anti-macrophage antibodies (0.5 μg: p=0.1, 5 μg: p<0.001; magnification 200×).

Panel B: Quantitative assessment of increase in foam cells, expressed as percentage of total area.

FIG. 6 shows the effect of treatment of vein grafts with the C5a receptor antagonists HC and AcF.

Panel A: Representative cross-sections of control and treated vein grafts 28 days after surgery; a decrease in intimal hyperplasia is seen in the groups treated with either AcF or HC at a dose of 0.3 mg/kg/day (haematoxylin-phloxine-saffron staining, magnification 200×).

Panel B: Quantification of intimal hyperplasia surface, expressed in mm², in control and treated vein grafts 28 days after surgery (n=7 per group; *represents p<0.05); C5a receptor antagonist treatment results in a decrease in intimal hyperplasia in the vein graft 28 days after surgery.

Panel C: Quantification of macrophages-derived foam cells contribution in intimal hyperplasia by immunohistochemistry, expressed as percentage of total intimal hyperplasia surface; treatment with C5a receptor antagonists results in a decrease in foam cell content in the vessel wall (n=7 per group, *represents p<0.05).

### DETAILED DESCRIPTION OF THE INVENTION

In a first aspect, the invention provides a method of prevention or treatment of intimal hyperplasia in blood vessel walls, comprising the step of administering a therapeutically effective amount of an inhibitor of C5a function to a mammal.

In one embodiment the mammal is the recipient of a blood vessel graft or prosthesis. The graft may be a venous or arterial graft. In another embodiment, the mammal is the recipient of an organ transplant such as a heart, heart-lung or kidney transplant.

In a second aspect, the invention provides a method of inhibiting the development of blood vessel graft thickening, comprising the step of administering a therapeutically effective amount of an inhibitor of C5a function to a mammal.

The graft may be a venous or arterial graft.

In a third aspect, the invention provides a method of inhibiting the development of stent restenosis, comprising the step of administering a therapeutically effective amount of an inhibitor of C5a function to a mammal into which a stent has been implanted.

In a fourth aspect, the invention provides a method of preventing or treating stenosis, restenosis or unwanted proliferation, migration or hypertrophy of cells in blood vessel walls or other anatomical structures of a mammal, comprising the step of administering a therapeutically effective mount of an inhibitor of C5a function to the mammal.

The stenosis, restenosis or unwanted proliferation, migration or hypertrophy of cells in blood vessel walls or other anatomical structures of a mammal may be a result of one of the following list of conditions; atherosclerosis, chronic obstructive pulmonary disorder, transplantation, vascular graft, venous surgery, arterial surgery, bypass graft failure, plastic surgery, tissue grafting, tumours, macular degeneration, neovascularisation, aberrant wound repair, endometriosis, vasculitis, defective revascularisation following thrombosis, prosthetic surgery, scarring, aneurysm surgery/repair, lymphatic surgery/repair, spinal injury/surgery/repair, endothelial tumours, cheloids, granulomas, haemangiomas, treatment/repair of post thrombotic disorders, angioplasty and reconstruction procedures.

In one embodiment the inhibitor is on or in an implantable intraluminal device, and the compound is administered by implanting the device within the mammal's body so that the compound elutes from the implanted device.

The device may comprise a stent, and may be implanted in an artery or vein of the mammal so that a therapeutically effective amount of the compound elutes from the stent and deters reocclusion of the artery or vein in which the stent is implanted. The artery may be a coronary artery.

In one embodiment, the compound is administered to a patient who has undergone or will undergo an angioplasty, atherectomy and/or stent implantation to treat an occluded blood vessel, and the compound is administered in an amount and by a route of administration which is effective to deter reocclusion of the blood vessel.

In another embodiment, the compound may be used to prevent the development of atherosclerosis in a subject at elevated risk of this condition, such as a smoker or a subject suffering from hypertension, metabolic syndrome or diabetes.

In one embodiment of each of these aspects of the invention, the inhibitor of C5a function is an antagonist of the C5a receptor (C5aR). In another embodiment the inhibitor of C5a function is an antibody, preferably a monoclonal antibody, directed against C5a. In a third embodiment the inhibitor of C5a function is a fragment of C5a.

Preferably the C5a receptor antagonist is a cyclic peptide or peptidomimetic compound of Formula I

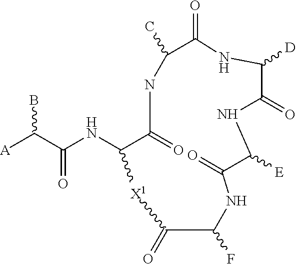

where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl.

B is an alkyl, aryl, phenyl, benzyl, naphthyl or indole group, or the side chain of a D- or L-amino acid such as L-phenylalanine or L-phenylglycine, but is not the side chain of glycine, D-phenylalanine, L-homophenylalanine, L-tryptophan, L-homotryptophan, L-tyrosine, or L-homotyrosine;

C is a small substituent, such as the side chain of a D-, L- or homo-amino acid such as glycine, alanine, leucine, valine, proline, hydroxyproline, or thioproline, but is preferably not a bulky substituent such as isoleucine, phenylalanine, or cyclohexylalanine;

D is the side chain of a neutral D-amino acid such as D-Leucine, D-homoleucine, D-cyclohexylalanine, D-homocyclohexylalanine, D-valine, D-norleucine, D-homo-norleucine, D-phenylalanine, O-tetrahydroisoquinoline, D-glutamine, D-glutamate, or D-tyrosine, but is preferably not a small substituent such as the side chain of glycine or D-alanine, a bulky planar side chain such as D-tryptophan, or a bulky charged side chain such as D-arginine or D-Lysine;

E is a bulky substituent, such as the side chain of an amino acid selected from the group consisting of L-phenylalanine, L-tryptophan and L-homotryptophan, or is L-1-napthyl or L-3-benzothienyl alanine, but is not the side chain of D-tryptophan, L-N-methyltryptophan, L-homophenylalanine, L-2-naphthyl L-tetrahydroisoquinoline, L-cyclohexylalanine, D-leucine, L-fluorenylalanine, or L-histidine;

F is the side chain of L-arginine, L-homoarginine, L-citrulline, or L-canavanine, or a bioisostere thereof, i.e. a side chain in which the terminal guanidine or urea group is retained, but the carbon backbone is replaced by a group which has different structure but is such that the side chain as a whole reacts with the target protein in the same way as the parent group; and

X is —(CH₂)ₙNH— or (CH₂)ₙ—S—, where n is an integer of 1, 2, 3 or 4, preferably 2 or 3; —(C₂)₂O—; —(CH₂)₃—; —(CH₂)₃—; —(CH₂)₄—; —CH₂COCHRNH—; or —CH₂—CHCOCHRNH—, where R is the side chain of any common or uncommon amino acid.

In C, both the cis and trans forms of hydroxyproline and thioproline may be used.

Preferably A is an acetamide group, an aminomethyl group, or a substituted or unsubstituted sulphonamide group.

Preferably where A is a substituted sulphonamide, the substituent is an alkyl chain of 1, 2, 3, 4, 5 or 6 carbon atoms, preferably of 1, 2, 3 or 4 carbon atoms, or a phenyl or toluoyl group.

In a particularly preferred embodiment, the compound has antagonist activity against C5a receptors, and has substantially no C5a agonist activity.

The compound is preferably an antagonist of C5a receptors on human and mammalian cells, including, but not limited to, human polymorphonuclear leukocytes, monocytes, lymphocytes and macrophages. The compound preferably binds potently and selectively to C5a receptors, and more preferably has potent antagonist activity at sub-micromolar concentrations. Even more preferably the compound has a receptor affinity IC50<25 μM, and an antagonist potency IC50<1 μM

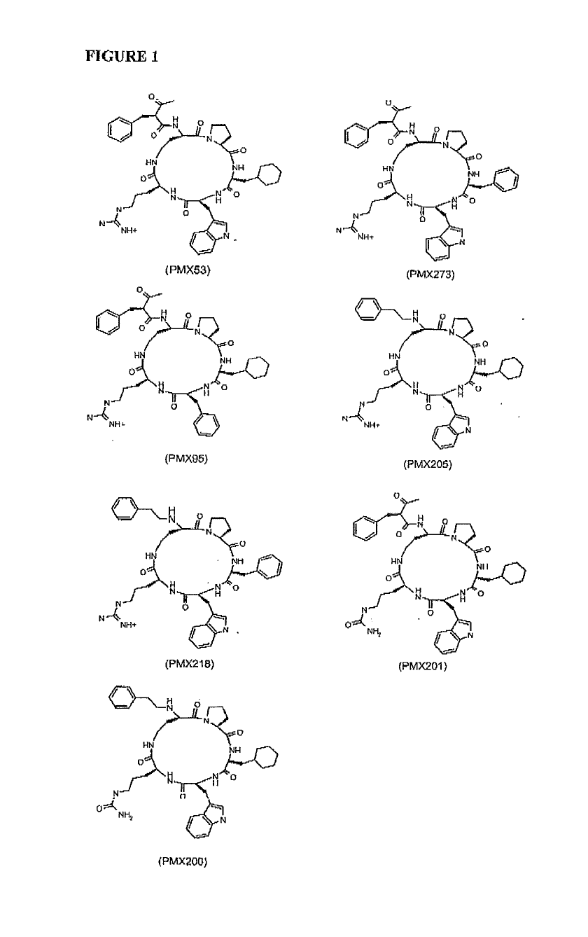

Most preferably the compound is compound 1 (PMX53; AcF└OP -DCha-WR┐), compound 33 (PMX273; AcF[OP-DPhe-WR]), compound 60 (PMX95; A cF[OP-DCha-FR]) or compound 45 (PMX201; AcF[OP-DCha-WCit]) described in International Patent Application No. PCT/AU02/01427 (WO 2003/033528), hydrocinnamate-[OPdChaWR](PMX205) or hydrocirmamate-└OPFdPheWR┐(PMX218). The structures of these cyclic peptides are illustrated in FIG. 1.

In a particularly preferred embodiment the C5a receptor antagonist is AcF-[OP-(D-Cha)WR] or hydrocinnamate-[OP-(D-Cha)WR].

Other C5a receptor antagonists are known; see for example the review by Sumichika (2004)Current Opinion in Investigational Drugs 5(5): 5 05-510; Sumichika et al. (2002) J Biol Chem, 277(51): 49403-49407; and International patent publications No. WO02/49993 by Neurogen Corporation and No. WO03/078457 by IBA GmbH. Many other small molecule non-peptidic inhibitors of C5a have been described, and the person skilled in the art will readily be able to identify relevant publications and patent specifications. U.S. Pat. No. 5,807,824 by Ciba-Geigy discloses polypeptide analogues of human C5a which are C5a receptor antagonists, dimeric forms of these analogues, and antibodies to the polypeptides. U.S. Pat. No. 5,480,974 by The Scripps Research Institute discloses monoclonal antibodies which bind to a 21-amino acid peptide found in the extracellular hydrophilic region of the human C5a receptor, and which blocks the activity of C5a. Pexeluzimab and eculizimab are monoclonal antibodies directed against C5, and are produced by Alexion Pharmaceuticals, Inc. Pexeluzimab is a humanised single chain antibody fragment. Both of these products are in clinical trials.

The inhibitor of C5a function may be administered systemically by any convenient parenteral route, for example by subcutaneous, intravenous or intramuscular injection. Some C5a receptors are available via enteral administration, and because of its convenience this route of administration may be preferable. The preferred compounds of the present invention may be administered orally or rectally.

Alternatively the inhibitor of C5a function may be administered locally to the site of an implant or prosthesis via a catheter or similar intravascular delivery device.

There are certain limitations associated with vascular surgery, an overgrowth of muscle cells can occur in the wall of the otherwise healthy blood vessels following access graft surgery (i.e. intimal hyperplasia). This is a significant problem as it can cause a complete blockage (de novo stenosis) of the blood vessel which usually results in the need for further surgery to avoid serious complications.

Further, patients who have kidney failure require their blood to be filtered through a dialysis machine to prevent them from dying. The process is normally carried out at least twice a week and involves the insertion of two needles into the patient—one to extract their blood and one to return it once it has been filtered. However, normal blood vessels cannot tolerate large needles being inserted into them repeatedly. One way to overcome this is to surgically insert a plastic tube between a vein and an artery in the patient's arm (“access graft”). Needles can then be repeatedly inserted into the graft to connect the patient to the dialysis machine.

However, up to 60 percent of haemodialysis access grafts block within one year of being inserted due to de novo stenosis, so that repeat surgery must be performed. Such repeat surgery also frequently fails, but more rapidly as less suitable sites are used, and can only be performed a limited number of tithes. Alternative and more difficult routes to achieve filtration are then required. In these circumstances, the life expectancy of patients can be short.

Accordingly, in another alternative embodiment, the inhibitor of C5a function may be releasably bound to the surface of a vascular graft, or an intraluminal medical device such as a stent or an endograft preventing intimal hyperplasia. The vascular graft may be an arterial or venous graft.

Methods for producing drug-eluting stents and the like are well known; see for example U.S. Pat. No. 6,358,556 by Roston Scientific Corporation, U.S. Pat. No. 5,545,208 by Medtronic, Inc., and U.S. Pat. No. 6,273,913 by Cordis Corporation. Drug-eluting stents are marketed by Cordis Corporation (the Cypher stent, which releases rapamycin) and Boston Scientific (the Taxus stent, which releases paclitaxel), and other such stents are being developed by Medtronic, Inc.

In particular, U.S. Pat. No. 6,140,127 by Cordis Corporation discloses methods for coating of stents with a peptide agent.

The mammal may be a human, or may be a domestic, companion or zoo animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as non-human primates, felids, canids, bovids, and ungulates.

We hypothesized that C5a plays a pro-inflammatory, prostenotic role in vein graft disease. If this hypothesis is correct, interference in C5a function should lead to a reduction in intimal hyperplasia and a decreased number of macrophage-derived foam cells in the intimal hyperplasia. To test the hypothesis, we used a mouse model for vein graft disease, in which vein graft thickening is accompanied by intimal hyperplasia and accelerated atherosclerosis. When vein grafts are performed in hypercholesterolemic ApoE−/− mice, the graft morphology 28 days after surgery strongly resembles the changes seen in diseased human vein grafts, making this model extremely useful to study the clinical problem of vein graft thickening (Zwolak et al. (1987)J Vasc Surg. 5: 126-136).

The presence and expression of C5 and its receptor (C5aR) was assessed in this mouse model. To examine the effect of C5a exposure on vein graft thickening, recombinant C5a was applied to the vein graft. The effect of abrogating C5a function was tested by treating mice which underwent vein graft surgery with two potent C5a receptor antagonists, AcF-[OP-(D-Cha)WR] and hydrocinnamate-[OP-(D-Cha)WR]). We have previously demonstrated that these compounds are able to inhibit C5a function in vivo; see for example International patent applications No. PCT/AU98/00490 (WO 99/00406) and No. PCT/AU02/01427 (WO 2003/033528); Finch et al. (1999) J Med. Chem. 42: 1965-1974; Woodruff et al. (2003)J. Immunol. 171: 5514-5520; Woodruff et al. (2004) J Surg Res. 116: 81-90, the entire contents of which are incorporated herein by this reference.

As used herein, the singular forms “a”, “an”, and “the” include the corresponding plural reference unless the context clearly dictates otherwise. Thus, for example, a reference to “an enzyme” includes a plurality of such enzymes, and a reference to “an amino acid” is a reference to one or more amino acids.

Where a range of values is expressed, it will be clearly understood that this range encompasses the upper and lower limits of the range, and all values in between these limits.

Intimal hyperplasia, also known as neointimal hyperplasia, is the abnormal migration and proliferation of vascular smooth muscle cells in the intimal layer of blood vessels, with associated deposition of extracellular connective tissue matrix.

Angioplasty, also known as percutaneous transluminal coronary angioplasty (PTCA), is a procedure iii which a catheter-guided balloon is used to open a narrowed coronary artery, allowing for improved flow of blood. A stent is usually placed at the narrowed section during angioplasty. Angioplasty may be used to treat coronary artery disease or peripheral artery disease of the legs. Angioplasty of the coronary artery, also known as percutaneous coronary intervention (PCI), together with placement of a stent is the first choice of treatment for a heart attack, if it can be performed in a timely manner, since it is less invasive and requires a shorter recovery time than bypass surgery, which is also done to increase blood flow to the heart muscle but requires open-heart surgery. In angioplasty of the aorta or the iliac arteries, a small expandable wire mesh tube called a stent is usually placed at the same time. Reclosure (restenosis) of the artery is less likely to occur if a stent is used. However, stents are typically not used with angioplasty of the femoral, popliteal, or tibial arteries, because these vessels are more subject to trauma and damage.

A stent is an expandable tube made of metal or plastic mesh which is inserted into a vessel or anatomical passage to keep its lumen open, and to prevent closure resulting from a stricture or external compression. Stents are usually inserted wider radiological guidance, and can be inserted percutaneously. Stents can be inserted into blood vessels to restore blood flow to the heart in people whose arteries have become dangerously narrowed due to atherosclerosis or other conditions. They are widely used in managing heart disease, and have become a standard part of practice in angioplasty. Stents are also commonly used in the oesophagus if it is narrowed by strictures or cancer, the ureters to maintain drainage from the kidneys, or the bile duct if it is narrowed by pancreatic cancer or cholangiocarcinoma.

Throughout the specification conventional single-letter and three-letter codes are used to represent amino acids.

For the purposes of this specification, the term “alkyl” is to be taken to mean a straight, branched, or cyclic, substituted or unsubstituted alkyl chain of 1, 2, 3, 4, 5, or 6, preferably 1, 2, 3 or 4 carbons. Most preferably the alkyl group is a methyl group. The term “acyl” is to be taken to mean a substituted or unsubstituted acyl of 1, 2, 3, 4, 5, or 6, preferably 1, 2, 3 or 4 carbon atoms. Most preferably the acyl group is acetyl. The term “aryl” is to be understood to mean a substituted or unsubstituted homocyclic or heterocyclic aryl group, in which the ring preferably has 5 or 6 members.

A “common” amino acid is a L-amino acid selected from the group consisting of glycine, leucine, isoleucine, valine, alanine, phenylalanine, tyrosine, tryptophan, aspartate, asparagine, glutamate, glutamine, cysteine, methionine, arginine, lysine, proline, serine, threonine and histidine.

An “uncommon” amino acid includes, but is not restricted to, D-amino acids, homo-amino acids, N-alkyl amino acids, dehydroamino acids, aromatic amino acids other than phenylalanine, tyrosine and tryptophan, ortho-, meta- or para-aminobenzoic acid, ornithine, citrulline, canavanine, norleucine, γ-glutamic acid, aminobutyrie acid, L-fluorenylalanine, L-3-benzothienylalanine, and α,α-disubstituted amino acids.

Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof; and/or may be therapeutic in terms of a partial or complete cure of a disease.

“Treating” as used herein covers any treatment of, or prevention of disease in a vertebrate, a mammal, particularly a human, and includes preventing the disease from occurring in a subject who may be predisposed to the disease, but has not yet been, diagnosed as having it, inhibiting the disease, i.e. arresting its development; or relieving or ameliorating the effects of the disease, i.e. cause regression of the effects of the disease.

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

Unless otherwise indicated, the present invention employs conventional chemistry, protein chemistry, molecular biological and enzymological techniques within the capacity of those skilled in the art. Such techniques are well known to the skilled worker, and are explained fully in the literature. See Coligan, Dunn, Ploegh, Speicher and Wingfield: “Current protocols in Protein Science” (1999) Volumes 1 and II (John Wiley & Sons Inc.); Sambrook, Fritsch and Maniatis: “Molecular Cloning: A Laboratory Manual” (2001); Shuler, M. L.: Bioprocess Engineering: Basic Concepts (2nd Edition, Prentice-Hall International, 1991); Glazer, A. N., DeLange, R. J., and Sigman, D. S.: Chemical Modification of Proteins (North Holland Publishing Company, Amsterdam, 1975); Graves, D. J., Martin, B. L., and Wang, J. II.: Co- and post-translational modification of proteins: chemical principles and biological effects (Oxford University Press, 1994) Lundblad, R. L. (1995) Techniques in protein modification. CRC Press, Inc. Boca Raton, Fla., USA; and Goding, J. W Monoclonal Antibodies: principles and practice (Academic Press, New York: 3^{rd} ed. 1996).

Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are described.

Zou et al. have described a method for performing venous bypass grafting in mice. In this model of venous bypass graft arteriosclerosis, arterialization of the venous graft predominantly caused by proliferation of vascular smooth muscle cells in normocholesterolemic C57BI/6 mice was described. This smooth muscle cell proliferation was significantly reduced in ICAM knockout mice, demonstrating the role of ICAM-1 in vein graft thickening (Zou et al (1998) Am J Pathol. 153: 1301-1310). In addition, pretreatment of the vein grafts with the growth factor receptor antagonist Suramin resulted in a significant reduction of neointimal hyperplasia, indicating that the PDGF receptor is involved in vein graft thickening (Hu et al, 1999).

Mouse models of arteriosclerosis, including transplant arteriosclerosis, are reviewed in Xu (2004) American Journal of Pathology 165(1): 1-10, and a model of accelerated atherosclerosis has been described by Lardenoye et al. (2002) Circulation Research 577-584.

Models for intimal hyperplasia have been described in rabbits (Zwolak et al, 1986; Alp et al (2002) Cardiovascular Research 56: 164-172) and in dogs (Petrofski et al. (2004) J Thorac Cardiovasc Surg. 127: 27-33), and these are also useful in further studies using the present invention.

### ABBREVIATIONS

Ac acetyl
AcF AcF-[OP-(D-Cha)WR]
bFGF basic fibroblast growth factor
C5aR C5a receptor
C5aRA C5a receptor antagonist
Cit citrulline
dCha D-cyclohexylamine
DPhe D-phenylalanine
HC hydrocinnamate-[OP-(D-Cha)WR]
IH intimal hyperplasia
PCA percutaneous coronary angioplasty
PDGF Platelet-derived growth factor
PG Pluronic gel
RT-PCR reverse-transcriptase polymerase chain reaction
VSMC vascular smooth muscle cells

The invention includes the use of various pharmaceutical compositions useful for ameliorating disease. The pharmaceutical compositions according to one embodiment of the invention are prepared by bringing a compound of formula I, analogue, derivatives or salts thereof and one or more pharmaceutically-active agents or combinations of compound of formula I and one or more pharmaceutically-active agents into a form suitable for administration to a subject using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in die amounts useful for in situ administration of the pharmaceutical composition, and animal models may be used to determine effective dosages for treatment of the cytotoxic side effects. Various considerations are described, e.g. in Langer, Science, 249: 1527, (1990).

Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be

(a) naturally occurring phosphatide such as lecithin;
(b) a condensation product of an alkylene oxide with a fatty acid, for example, polyoxyethylene stearate;
(c) a condensation product of ethylene oxide with a long chain aliphatic alcohol, for example, heptadecaethylenoxycctanol;
(d) a condensation product of ethylene oxide with a partial ester derived from a fatty acid and hexitol such as polyoxyethylene sorbitol monooleate, or
(e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation of injectables.

Compounds of formula I may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the compound of formula I of the present invention will usually be of the order of about 0.5 mg to about 20 mg per kilogram body weight, with a preferred dosage range between about 0.5 mg to about 10 mg per kilogram body weight per day (from about 0.5 g to about 3 g per patient per day), more preferably 0.1 mg/kg to 10 mg/kg per day. The amount of active ingredient which may be combined with the carrier materials to produce a single dosage will vary, depending upon the host to be treated and the particular mode of administration. For example, a formulation intended for oral administration to humans may contain about 5 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other therapeutic compounds to provide an operative combination. It is intended to include any chemically compatible combination of pharmaceutically-active agents, as long as the combination does not eliminate the activity of the compound of this invention. For example, the compounds of the invention may be administered in conjunction with any agent known to be beneficial in the treatment or prevention of intimal hyperplasia. This includes oral, rectal or parenteral administration in conjunction with implantation of a drug-coated stent. In order that the nature of the present invention may be more clearly understood, preferred forms thereof will now be described with reference to the following non-limiting examples.

### Example 1

### General Methods

### Peptide Synthesis

Cyclic peptide compounds of formula I are prepared according to methods described in detail in our earlier applications No. PCT/AU98/00490 (WO 99/00406) and No. PCT/AU02/01427 (WO 2003/033528), the entire disclosures of which are incorporated herein by this reference. While the invention is specifically illustrated with reference to the compounds AcF-[OPdChaWR] (PMX53), whose corresponding linear peptide is Ac-Phe-Orn-Pro-dCha-Trp-Arg, and to hydrocinnamate-[OP-(D-Cha)WR](PMX205) whose corresponding linear peptide is hydrocinnamate-Orn-Pro-dCha-Trp-Arg, it will be clearly understood that the invention is not limited to these compounds.

Compounds 1-6, 17, 20, 28, 30, 31, 36 and 44 disclosed in International patent application No. PCT/AU98/00490 (WO 99/00406) and compounds 10-12, 14, 15, 25, 33, 35, 40, 45, 48, 52, 58, 60, 66, and 68-70 disclosed for the first time in International patent application PCT/AU02/01427 (WO 2003/033528) have appreciable antagonist potency (IC50<1 μM) against the C5a receptor on human neutrophils. PMX205, PMX53, and PMX273, PMX201 and PMX218 are most preferred.

We have found that all of the compounds of formula I which have so far been tested have broadly similar pharmacological activities, although the physicochemical properties, potency, and bioavailability of the individual compounds varies somewhat, depending on the specific substituents.

The general tests described below may be used for initial screening of candidate inhibitors of C5a receptors.

### Drug Preparation and Formulation

The human C5a receptor antagonists AcF-[OPdChaWR] (AcPhe[Orn-Pro-D-Cyclohexylalanine-Trp-Arg]) and hydrocinnamate-[OP-(D-Cha)WR] were synthesized as described above, purified by reversed phase HPLC, and fully characterized by mass spectrometry and proton NMR spectroscopy. The C5a antagonists were prepared in 30% propylene glycol for subcutaneous injection.

### Receptor Binding Assay

Assays are performed with fresh human PMNs, isolated as previously described (Sanderson, et al, 1995) using a buffer of 50 mM HEPES, 1 mM CaCl₂, 5 mM MgCl₂, 0.5% bovine serum albumin, 0.1% bacitracin and 100 μM phenylmethylsulfonyl fluoride (PMSF). In assays performed at 4° C., buffer, unlabelled human recombinant C5a (Sigma) or test peptide, labelled ¹²⁵I-C5a (˜20 pM) (New England Nuclear, MA) prepared by the Hunter/Bolton method and PMNs (0.2×10⁶) are added sequentially to a Millipore Multiscreen assay plate (HV 0.45) having a final volume of 200 μL/well. After incubation for 60 min at 4° C., the samples are filtered and the plate washed once with buffer. Filters are dried, punched and counted in an LKB gamma counter. Non-specific binding is assessed by the inclusion of 1 mM peptide or 100 nM C5a, which typically results in 10-15% total binding.

Data are analysed using non-linear regression and statistics with Dunnett post-test.

### Myeloperoxidase Release Assay for Antagonist Activity

Cells are isolated as previously described (Sanderson et al, 1995) and incubated with cytochalasin B (5 μg/mL, 15 min, 37° C.). Hank's Balanced Salt solution containing 0.15% gelatin and test peptide is added on to a 96 well plate (total volume 100 μL/well), followed by 25 μL cells (4×10⁶/mL). To assess the capacity of each peptide to antagonise C5a, cells are incubated for 5 min at 37° C. with each peptide, followed by addition of C5a (100 nM) and further incubation for 5 min. Then 50 μL of sodium phosphate (0.1 M, pH 6.8) is added to each well, the plate was cooled to room temperature, and 25 μL of a fresh mixture of equal volumes of dimethrocybenzidine (5.7 mg/mL) and H₂O₂ (0.51%) is added to each well. The reaction is stopped at 10 min by addition of 2% sodium azide. Absorbances are measured at 450 nm in a Bioscan 450 plate reader, corrected for control values (no peptide), and analysed by non-linear regression.

### Materials and Methods

### Mice

All experiments were approved by the Animal Welfare Committee of the Department of Surgery, Leiden University Medical Centre. Male ApoE^{-/-} mice, aged between 12 and 16 weeks, were used for all experiments. Mice were fed a standard chow diet, and received water and food ad libitum. Before surgery, mice were anesthetized by an intra-peritoneal injection with Midazolam (5 mg/kg, Roche), Medetomidine (0.5 mg/kg, Orion) and Fentanyl (0.05 mg/kg, Janssen). Cholesterol levels in serum were determined at the time of sacrifice.

### Vein Graft Model

Vein graft surgery was performed as described by Zou et al (1998) Am J. Pathol. 153: 1301-1310. Briefly, caval veins were harvested from genetically identical donor mice, and were preserved before implantation at 4° C. in 0.9% NaCl containing 100 U/ml of heparin. In the graft recipient, the right carotid artery was dissected and cut in the middle. A polyethylene cuff was placed at both ends of the artery. The artery was everted around the cuff and ligated with a silk 8.0 suture. The caval vein was sleeved over the two cuffs and ligated. Successful engraftment was confirmed by the presence of pulsations and turbulent blood flow within the vein graft. The whole procedure usually takes about 30 minutes.

### Detection of C5 in Vein Grafts by Immunohistochemistry

Twenty-four mice were sacrificed after vein graft surgery at the following time points: directly after surgery, 24 h, 3 d, 7 d, 14 d and 28 d after surgery. At the time of sacrifice, vein grafts were harvested after 5 minutes of in vivo perfusion fixation with 4% formaldehyde. Vein galls were fixed overnight in 4% formaldehyde, dehydrated and embedded in paraffin. Serial 5 μm perpendicular cross-sections of the embedded vessel were made through the entire specimen. Antibodies directed against murine C5 (HyCult Biotechnology; 1:25 dilution) were used to detect the presence of C5 in vein grafts by immunohistochemistry. The person skilled in the art will be aware that C5a is a cleavage product of C5, and that if C5a is produced this will occur at the sites at which C5 is identified.

RNA Isolation, cDNA Synthesis and RT-PR

Total RNA was isolated from 16 vein grafts harvested 24 after surgery and at 3, 7, and 28 days after surgery, and from caval veins of donor mice.

An RNA Isolation Mini Kit for Fibrous Tissue (Qiagen) was used to isolate RNA, following the protocol provided by the manufacturer. To avoid DNA contamination, a DNase treatment was included (RNase Free DNase set, Qiagen). RNA (250 μg) was reverse-transcribed using the Ready-To-Go You-Prime First-Strand Beats kit (Amersham Biosciences) according to the manufacturer's protocol.

Semi-quantitative RT-PCR (Robocycler Gradient 96, Stratagene) was performed with primers for C5a receptor:

Forward: GACCCCATAGATAACAGCA
Reverse: CAGAGGCAACACAAAACCCA

(Van Beck et al (2000) Exp Neurol. 161: 373-382) and β-actin (Perkin-Elmer).

Samples were amplified for 35 cycles following an initial cycle for 2 min. at 94° C. Each cycle consisted of 30 sec. at 94° C., 30 sec at 56° C. and 90 sec. at 65° C., followed by an extension cycle of 4 min at 74° C. PCR products were visualized on a 1.2% agarose gel containing ethidium bromide.

### Quantification and Histological Assessment of Intimal Hyperplasia

Cross-sections of vein grafts were stained with hematoxylin-phloxine-saffron, using conventional methods. The intimal hyperplasia surface of the thickened vein grafts was measured using image analysis software (Qwin, Leica). For each vein graft, six equally spaced cross-sections were used to determine the degree of vessel wall thickening.

To identify macrophages and macrophage-derived foam cells, immunohistochemistry was performed using AIA31240 primary antibodies against mouse macrophages (1:3000, Accurate Chemical). The contribution of macrophages and foam cells to the intimal hyperplasia was measured by computer-assisted analysis (Qwin, Leica) as the area stained with antibody AIA31240, and expressed as a percentage of the total intimal hyperplasia surface.

### Statistical Analysis

All data are presented as mean±SEM. To determine statistical significance overall, comparisons between all groups were made using one-way ANOVA. Where differences were significant, each group was then separately compared to the control group, using Student's t test. P-values of <0.05 were regarded as significant.

### Example 2

### C5 is Present in Remodelling Vein Grafts

The presence of C5 in the developing intimal hyperplasia was assessed by immunohistochemistry in vein grafts (n=4 per time point), harvested at several time points after surgery (t=6 h, 1, 3, 7, 14 and 28 days).

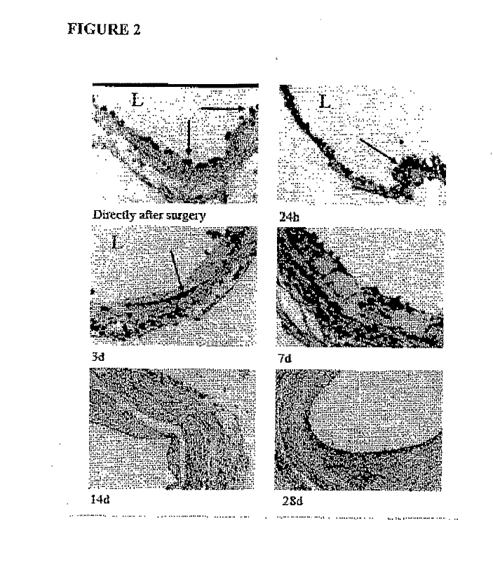

In the vein gratis harvested 6 hours and one day after surgery, a large amount of C5 could be detected in adhering monocytes and in adventitial fibroblasts. C5 was also detected in the regenerating endothelium as from 7 days after surgery. At this stage, staining appeared most pronounced and diffusely present, indicating the presence of high amounts of C5 in the vessel wall. At the later time points (14 and 28 days post-operatively), this was observed in parallel with development of intimal hyperplasia expression of C5 was seen in endothelial cells, adhering monocytes, adventitial fibroblasts and foam cells, Only small numbers of smooth muscle cells displayed positive staining for C5, as shown in FIG. 2.

### Example 3

### Time-Dependent Expression of C5a Receptor mRNA in Remodelling Vein Grafts

To assess whether the receptor for C5a was present in and produced in the vein grafts, total RNA was isolated and tested for the presence of C5aR mRNA by RT-PCR. Vein grafts were harvested at several time points (t-24 h, 3 d, 7 d and 28 d, n=4 per time point), and normal caval veins were also harvested to serve as controls. The amount of total cDNA in all specimens was assessed by the presence of the housekeeping gene β-actin.

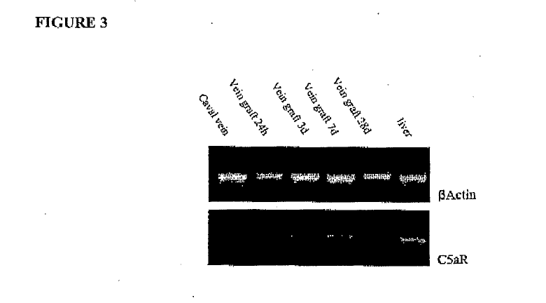

Normal caval veins showed minimal expression of C5aR. Expression of C5a in the vein grafts gradually increased in a lime-dependent fashion. Peak expression was seen at 7 days after surgery, after which expression declined to the level observed in normal caval vein at twenty-eight days after surgery; this is illustrated in FIG. 3.

These data confirm that the receptor for C5a is present in the vein graft, and is up-regulated in the early stages of the process of vein graft thickening.

### Example 4

### C5a Increases Intimal Hyperplasia

To examine the involvement of C5a in the development of vein graft thickening, we applied recombinant C5a in two concentrations (0.5 μg and 5 μg, dissolved in 100 μl 20% Pluronic gel, n=7 per group,) directly to the vein graft at the time of surgery. In the control group 100 μl of 20% Pluronic gel without C5a was applied. Serum cholesterol at the time of surgery was 10.4±1.2, and did not change significantly during the experiment. Body weight and scrum cholesterol did not differ between the three groups.

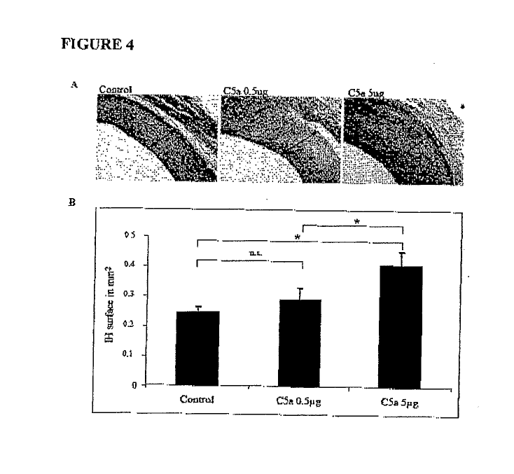

Vein grafts were harvested after 28 days, and vein graft thickening was quantified. Over-expression of C5a resulted in a dose-dependent increase of vein graft thickening caused by intimal hyperplasia, as shown in FIGS. 4A and 45. Treatment with recombinant C5a resulted in a dose-dependent increase of the intimal hyperplasia surface, compared to control vein grafts (control: 0.24±0.02 mm², 0.5 μg C5a: 0.29±0.03 mm² vs, p=0.14, 5 μg C5a: 0.41±0.04 mm², p=0.002 when compared to controls, p=0.037 when compared to the 0.5 μg C5a-treated group). No significant differences in luminal area were seen between the three different groups.

### Example 5

### C5a increases the foam cell content in intimal hyperplasia

Since C5a is a potent chemotactic factor for monocytes/macrophages, the numbers of macrophages and macrophage-derived foam cells in the thickened vein graft were studied.

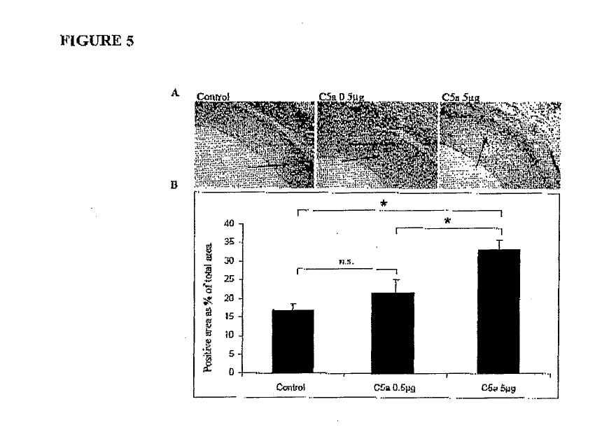

Twenty-one mice were randomly divided into three groups. In the treatment groups, either 0.5 μg or 5 μg of E. coli-derived recombinant mouse C5a (HyCult Biotechnology) was dissolved in 0.1 ml of 20% Pluronic gel, and applied around the venous interposition at the time of surgery. In the control group 0.1 ml of 20% Pluronic gel was applied without C5a. Mice were sacrificed after 28 days. A dose-dependent increase in the contribution of macrophages to the intimal hyperplasia was observed in the C5a-treated vein grafts, as shown in FIGS. 5A and 5B.

In the control group, approximately 17±2% of intimal hyperplasia consisted of macrophages and macrophage-derived foam cells. When 0.5 μg C5a was applied, this percentage increased to 22±3% (p-0.11). Application of 5 μg C5a to the vein graft resulted in a significantly increased macrophage/foam cell content of 33±2% (p<0.001 when compared to control grafts; p=0.008 when compared to the grafts treated with 0.5 μg C5a).

### Example 6

### Treatment with C5a Receptor Antagonists Reduces Intimal Hyperplasia

In order to inhibit C5a function, 21 mice were randomly divided in to three groups, and were treated with the cyclic peptide C5a antagonists AcF-[OP-(D-Cha)WR] (AcF) or hydrocinnamate-[OP-(D-Cha)WR] (HC). Both compounds display potent antagonizing activity for the C5a receptor, and were synthesized as described above and in March et al. (2004) Mol. Pharmacal. 65: 868-879.

AcF was administered subcutaneously in a daily dose of 3 mg/kg in 0.1 ml 30% propylene glycol and 70% sterile water, starting one day prior to surgery. Since HC displays an increased in vivo potency over AcF, in a dose-dependent fashion (Woodruff et al. (2005) J Pharmacal Exp Ther.), it was administered in 2 dosages, 3 mg/kg/day and 0.3 mg/kg/day, both in 0.1 ml 30% propylene glycol and 70% sterile water. A control group (n=7) received daily injections of 0.1 ml 30% propylene glycol and 70% sterile water. All mice were sacrificed 28 days after surgery.

Twenty-eight mice received either the C5a receptor antagonist AcF in 30% propylene glycol (3 mg/kg/day subcutaneously (s.c.), n=7), the C5a receptor antagonist HC in 30% propylene glycol (3 and 0.3 mg/kg/day s.c., n=7 each), or daily injections of 30% propylene glycol s.c. (n=7). Serum cholesterol at the time of surgery was 11.7±2.0. No differences were observed in body weight or scrum cholesterol between the different treatment groups.

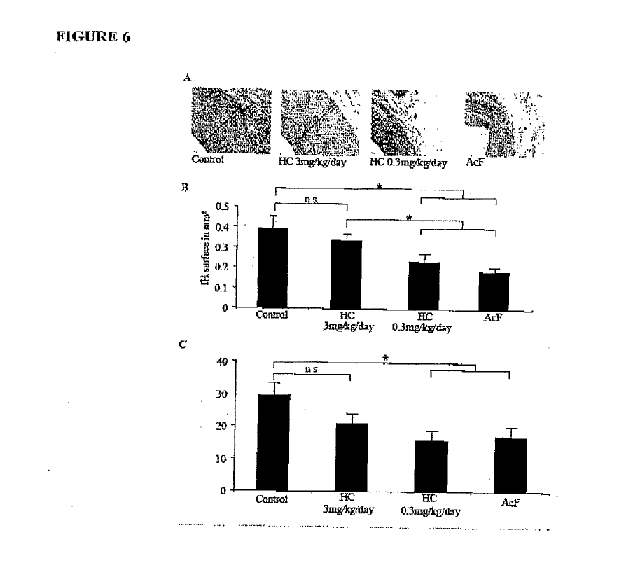

Treatment with AcF resulted in a significant decrease in vein graft thickening of 53% when compared to the control mice (0.19±0.03 mm², control: 0.39±0.06 mm²; p=0.046), whereas treatment with a similar dose of 11 C did not result in decreased vein graft thickening (0.33±0.03 mm², p=0.23). However, treatment with a 10-fold lower dose of 0.3 mg/kg HC did lead to significantly reduced intimal hyperplasia when compared to the control animals (0.23±0.03 mm², p=0.035). These results are summarised in FIGS. 6A and 6B.

No differences in luminal size were seen between the control group, AcF and 3 mg/kg HC, whereas treatment with 0.3 mg/kg HC did result in a significantly increased luminal area (control: 0.42±0.04 mm²; AcF: 0.41±0.08 mm², p=0.48; HC 3 mg/kg: 0.48±0.03 mm², p=0.15; HC 0.3 mg/kg: 0.61±0.04 mm², p=0.005).

### Example 7

### C5aRA Treatment Diminishes Foam Cell Content in Intimal Hyperplasia

Intimal hyperplasia of control, untreated vein grafts consisted of approximately 30±4% foam cells. A significant reduction in this foam cell contribution was seen in the AcF-treated vein grafts (16±3%, p=0.01 when compared to controls). No significant reduction was seen in the group treated with HC at 3 mg/kg/day (22±3% foam cells in intimal hyperplasia, p=0.07 when compared to controls). However, administration of HC at a daily dose of 0.3 mg/kg/day did result in a significantly lowered contribution of foam cells in the intimal hyperplasia (16±3%, p=0.01). These results are summarised in FIG. 6C.

### Example 8

### Effect of C5aRa in Models of Atherosclerosis

For further investigation of the effects of C5a receptor antagonists on atherosclerosis cellular models using rat vascular smooth muscle cells (VSMC), endothelial cells and macrophages for in vitro studies and a transgenic mouse model (ApoE Knockout) for in vivo studies may be used. A rat restenosis model and a rabbit denudation/hypercholesteremia model of atherosclerosis are also available.

PMX C5a receptor antagonists are initially used for in vitro studies to determine their effects on proliferation of VSMC alone and in co-culture with endothelial cells and/or macrophages.

Compounds are also tested in vivo models of disease, such as ApoE Knockout mouse model, the rabbit denudation/hypercholesteremia model, and the rat restenosis model. The compounds are administered in both prophylactic and therapeutic protocols.

### Example 9

### Discussion

We have demonstrated for the first time the involvement of the complement component C5a in the process of vein graft thickening. C5a is a highly potent chemotactic agent for immune and inflammatory cells, including monocytes, neutrophils and T-cells, and has been shown to modulate pro-inflammatory effects in several diseases, but its role in vein graft thickening has never been previously assessed.

In this study, the presence of C5 protein in vein grafts was demonstrated by immunohistochemistry at several time points after surgery. C5 was predominantly expressed in adhering monocytes, adventitial fibroblasts, endothelial cells and foam cells in the intimal hyperplasia, and staining appeared to be most intense 7 days after surgery. However, since the antibody used was not specific for C5a, this could have been in the form of C5, C5a and C5b-9.

In addition, the time course of expression of mRNA encoding the C5a receptor was followed by RT-PCR. At baseline, in the normal caval vein, very minimal levels of C5aR mRNA were detected. A fast up-regulation was then observed in the first days after engraftment of the vein. Peak expression occurred after 7 days after surgery, coinciding with the highest levels of C5 staining seen using immunohistochemistry. Expression subsequently declined to baseline levels 28 days after engraftment.

In order to study the functional involvement of C5a in the formation of intimal hyperplasia, the effect of increased exposure to C5a was studied by applying murine recombinant C5a protein to vein grafts. Since chemotaxis of inflammatory cells to the vein graft is one of the earliest phenomena seen in the process of intimal hyperplasia development, we hypothesized that increased exposure to C5a would result in increased intimal hyperplasia formation. We found not only that increased exposure to C5a aggravated intimal hyperplasia formation, but also increased macrophage-derived foam cell content in the intimal hyperplasia; both these responses to C5a were dose-dependent.

Moreover, when C5a function was blocked, using the potent C5aR antagonists AcF and HC, the opposite effect was seen. Inhibiting C5aR function resulted in decreased intimal hyperplasia development, with a reduced contribution of foam cells to the intimal hyperplasia.

These data indicate that there is a pro-stenotic, pro-atherogenic effect of C5a in this murine in vivo model for vein graft disease.

We found that the effect of HC was dose-dependent. When administered at the highest dose, 3 mg/kg/day, treatment with HC did not inhibit intimal hyperplasia in our model. This is in line with our results with HC in a rat model of inflammatory bowel disease (Woodruff et al. (2005) J Pharmacal Exp Ther.). We hypothesized that a lack of therapeutic effect on inflammatory bowel disease of HC administered in high dosages might be due to deleterious effects at unidentified receptors, to local-toxicity, or to other factors not yet recognized. Similar results were found in the present study, in which the higher dose of HC used, 3 mg/kg, was not effective, but intimal hyperplasia was significantly reduced when a 10-fold lower dose was administered.

The results of the present study, using two separate C5a receptor antagonists, clearly indicate a role for the C5aR in the pathogenesis of intimal hyperplasia.

Little is known about the role of C5a in other forms of inflammation-related vascular remodelling. Although there have been several studies which argue for a role of complement in spontaneous atherosclerosis, these studies report inconclusive results. Both C5 mRNA and protein are present in normal arteries, and there is a substantial increase in C5 mRNA and protein atherosclerotic arteries (Yasojima et al. (2001) Am J Pathol. 158: 1039-1051). ApoE/C5 double knock-out mice develop spontaneous atherosclerosis at a similar rate to their ApoE−/− littermates (Patel et al. (2001) Biochem Biophys Res Commun. 286: 164-170). However, C5 knock-out mice are not suitable for specific identification of the role of C5a, since C5 depletion also inhibits the formation of complement components C5b-9. Increased serum levels of C5a in patients with advanced atherosclerosis were associated with increased cardiovascular risk, as determined by the occurrence of major adverse cardiovascular events (Speidl et al. (2005) Eur Heart J.) suggesting that C5a might be a valuable marker for risk assessment in patients.

Hitherto there have been no reports of a role of C5a in a third form of vascular remodelling, namely post-angioplasty restenosis. Although these lesions usually consist mainly of smooth muscle cells in the neointima and only few inflammatory cells are present, it has been shown that inflammation plays a pivotal role in this process (Danenherg et al. (2002) Circulation. 105: 2917-2922; Toutouzas et al. (2004) Eur Heart J. 25: 1679-1687). Moreover, other chemotactic factors have been shown to be involved in the development of restenosis (Chen et al, (2004) Arterioscler Thromb Vasc Biol. 24: 709-714; Usui et al. (2002) FASEB J. 16: 1838-1840).

Our results indicate that C5a is also likely to be involved in atherosclerosis and in restenosis, and therefore that C5a receptor antagonists are also useful in the treatment and prevention of these conditions.

References cited herein are listed on the following pages, and are incorporated herein by this reference.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context liar the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of prevention or treatment of intimal hyperplasia in blood vessel walls, the method comprising the step of administering a therapeutically effective amount of an inhibitor of C5a function to a mammal.

2-41. (canceled)
